(19) United States
(12) Patent Application Publication
(10) Pub. No.: US 2019/0336065 A1
(43) Pub. Date: Nov. 7, 2019

(54) METHOD AND COMPUTER PROGRAM FOR MONITORING INCONTINENCE

(71) Applicant: The Procter & Gamble Company, Cincinnati, OH (US)

(72) Inventors: Eric Daniel Ricciardi, Cincinnati, OH (US); Sion Agami, Mason, OH (US); Alexzandra Joan Ramachandran, Cincinnati, OH (US)

(21) Appl. No.: 16/405,453

(22) Filed: May 7, 2019

Related U.S. Application Data
(60) Provisional application No. 62/667,958, filed on May 7, 2018.

(51) Int. Cl. A61B 5/00; G06Q 30/06; G16H 40/63; A61B 5/20

(52) U.S. Cl. CPC A61B 5/486; G06Q 30/0631; A61B 5/742; A61B 5/202; A61B 5/7275; G16H 40/63

(57) ABSTRACT

A method for managing urinary incontinence is provided. The method includes receiving historical information through an application; receiving input through an application; and making a recommendation to the user regarding an absorbent article.

### CROSS REFERENCE TO RELATED APPLICATION

This application claims the benefit of U.S. Provisional Application No. 62/667,958, filed May 7, 2018, the substance of which is incorporated herein by reference.

### FIELD OF INVENTION

The present invention relates to a method for monitoring incontinence, a mobile device for carrying out the method, and a computer program for causing the mobile device to carry out the method.

### BACKGROUND OF THE INVENTION

Urinary and/or fecal incontinence causes many people to use various types of absorbent products, such as incontinence pads, diapers etc. Often, the people affected by urinary and/or fecal incontinence begin to reduce the distance from home they are willing to travel or the activities they attend. Many may choose to stay home versus taking the risk of an incident outside of the home. The person may feel that their accidents are random and therefore is not willing to go outside the home or venture beyond a certain distance thereby effectively reducing their world.

Additionally, the person may feel that their individual scenario is beyond “normal” without fully knowing what “normal” may be or how they compare to others. Essentially, because the person has a data set of one, they do not know if their incontinence is within the average for their cohort, below the average for their cohort, or beyond the average for their cohort. To many people suffering from incontinence, this is a huge problem often causing feelings of shame and humiliation and sometimes even social withdrawal and isolation.

Monitoring and timely change of absorbent products may sometimes be difficult when it comes to adults who due to the inability to control the urinary or fecal function finds it difficult to know when voiding has taken place and hence when to change the absorbent product. Additionally, monitoring historical data to give a user predictions and recommendations on how to manage their incontinence is hard due to a lack of centralized data and because many users do not want others to know of their incontinence issues.

However, known solutions for monitoring use of absorbent articles often involve complex and expensive products and/or monitoring systems that are not readily available to the public. Additionally, as previously stated, there is no current collection or aggregate of historical data in any one place for a single user or for persons with incontinence as a whole. As such, a system that allows a user to monitor their incontinence during different days and activities is needed. Preferably, a system that helps the user plan ahead based on their prior data and the data of those in their cohort such that the user may expand their lifespace beyond that which they have chosen without the use of the needed system.

### SUMMARY OF THE INVENTION

It is an object of the present invention to solve or at least mitigate one or more of the above-mentioned problems.

In particular, it is an object of the present invention to provide a cost efficient and readily available method for monitoring a user's incontinence and the user's use of absorbent products under one or more daily conditions such that the user may use the monitoring system to make decisions regarding their lifespace and their absorbent article usage.

Another object of the invention is to provide a method that can help minimize incidents of incontinence. The method comprising at least the steps of obtaining information through the use of a mobile device; predicting, by the mobile device, based on the information, a future voiding by the wearer; providing, by the mobile device, product recommendations for a given activity.

Another object of the invention is to provide a method that can help minimize incidents of incontinence. The method comprises at least the steps of monitoring a user's bathroom habits and the use of an absorbent product; obtaining, by means of a mobile device, intake information indicative of intake of fluid and/or solid edible material by the wearer; obtaining, by means of one or more sensors, information regarding one or more of the user's environment, the user's daily previous activities, the user's calendar, or the user's biometric data, and providing product-recommendations for a future activity.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 depicts a computing environment, illustrating a system for data collection.

FIG. 2 depicts the basic principles of the method.

FIG. 3 depicts a personalized user curve.

FIG. 4 depicts a personalized Confidence Indicator.

### DETAILED DESCRIPTION OF THE INVENTION

As used herein, the term “comprising” means that the various components, ingredients, or steps, can be conjointly employed in practicing the present invention. Accordingly, the term “comprising” encompasses the more restrictive terms “consisting essentially of” and “consisting of ” Other terms may be defined as they are discussed in greater detail below.

As used herein, the term “computing device” refers to a system comprising an input device, a processor, at least one memory, and at least one output device coupled together via electrical circuitry or other suitable coupling means. Optionally, a network interface is also provided, for connecting a first computing device to a communications network.

Herein, the term “absorbent articles” refers to articles which absorb and contain body fluids or exudates, and more specifically, refers to articles which are placed against or in proximity to the body of the wearer to absorb and contain the various exudates discharged from the body. The term “disposable” is used herein to describe absorbent articles which are not intended to be laundered or otherwise restored or reused as an absorbent article (i.e., they are intended to be discarded after a single use, and preferably, to be disposed of in an environmentally compatible manner). Typical disposable feminine hygiene absorbent articles according to the present invention are sanitary napkins, panty liners, tampons, absorbent articles for adult incontinence or the like. Absorbent articles may also include articles for male incontinence. Feminine hygiene articles do not include baby diapers.

Herein, the term “feminine hygiene absorbent article” is used in a broad sense including any article able to receive and/or absorb and/or contain and/or retain body fluids/bodily exudates such as menses, vaginal secretions, and urine. Exemplary feminine hygiene absorbent articles include disposable feminine hygiene absorbent articles. Such articles include tampons, sanitary napkins, interlabial products, incontinence devices, and pantiliners.

As used herein, the term “input device” refers to a device used to interact with a computing device, for example, a computer mouse, electronic stylus (or “pen”), touch screen, keyboard, keypad, microphone/voice recognition system, scanner, or other suitable apparatus for entering information in electronic form known in the art. The input/output hardware may include and/or be configured to interface with a monitor, positioning system, keyboard, mouse, printer, image capturing device, microphone, speaker, gyroscope, compass, and/or other device for receiving, sending, and/or presenting data.

As used herein, the term “memory” refers to a volatile or non-volatile storage media, such as ROM, SRAM, DRAM, and/or other types of RAM, flash memory, secure digital (SD) memory, registers, compact discs (CD), digital versatile discs (DVD), and/or other types of non-transitory computer-readable mediums. As is well known in the art, memory may include a plurality of distributed memory coupled via electrical circuitry. These non-transitory computer-readable mediums may reside within the mobile computing device and/or external to the mobile computing device. Additionally, the memory component may be configured to store operating logic and an absorbent article application. The absorbent article application may include a plurality of different pieces of logic including the absorbent article identification logic, the absorbent article selection logic, and the real-time still or video image capturing and altering logic, each of which may be embodied as a computer program, firmware, and/or hardware, as an example.

As used herein, the term “mobile computing device” may include a mobile telephone, personal digital assistant, laptop computer, tablet, and/or other mobile device. A mobile computing device may have a memory that stores a computer application that causes the mobile computing device to prompt the individual to capture a real-time image of the absorbent article.

As used herein, the term “output device” refers to a computer monitor, LCD display, mobile device, tablet, flat-screen display (for example, gas-plasma, activematrix or LED display), or similar device (including a display of a personal digital assistant or other handheld or portable device) suitable for display of visual information, and/or a speaker or other suitable device for outputting audio information, as known in the art.

As used herein, the term “processor” relates to one or more suitable microprocessors well known in the art, which processes data and computing instructions received via input device or stored in memory. Processor executes computing instruction to perform processes such as those described below in accordance with the present invention. The processor may include any processing component operable to receive and execute instructions (such as from the data storage component and/or memory component). The input/output hardware may include and/or be configured to interface with a monitor, positioning system, keyboard, mouse, printer, image capturing device, microphone, speaker, gyroscope, compass, and/or other device for receiving, sending, and/or presenting data.

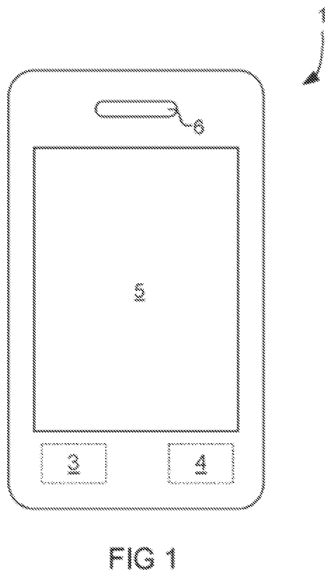

Referring now to the drawings, FIG. 1 illustrates a mobile device 1 for performing the method according to the invention. The mobile device 1 in FIG. 1 is a mobile phone in form of what is often referred to as a smartphone but it should be appreciated that the mobile device according to the invention may be any type of hand-held computing device, such as a personal digital assistant (PDA) or a tablet computer, devised and configured as set forth below. The mobile device 1 comprises a processor 3 for processing data. The data may be received from communication devices to which the mobile device 1 is communicatively connectable via a network, or stored on a digital storage medium 4 of the mobile device, which storage medium is accessible by the processor 3.

The mobile device 1 is further seen to comprise a display 5 for displaying information to a user, and, if realized in form of a touch-display, also for receiving information from the user in form of user input. The mobile device 1 may also comprise other means for receiving user input, such as buttons, microphones, etc. Furthermore, the mobile device 1 comprises a loudspeaker 6 for outputting sound signals to the user. The mobile device 1 is operable to perform all method steps of the inventive method, which method steps will be described in more detail below, through execution of a computer program stored in the storage medium 4.

Preferably, the computer program is realized in form of a stand-alone application, meaning that no data has to be received from application servers residing on a network to which the mobile device 1 must be connected. However, the computer program may also be a client application of a distributed software solution further comprising a server-side application residing in an application server to which the mobile device is communicatively connectable. In this case, some of the method steps described below may be performed by the application server through execution of the server-side application.

The method uses a mobile application to interface the user. The application may be loaded on more than one mobile device for a single user. Additionally, the user may give access to others so that they may see their individual account. In giving access to others, the user may choose the level of access given to each individual. For example, a user may allow a caretaker to have full access to both input, modify, and analyze data within the application. In contrast, a user may give access to their doctor to simply see results and analyze data versus being able to give input or modify the data. Additionally, a user may give purchasing power to an individual so that products are purchased on their behalf through the use of the application.

Data collected by a computing or mobile device is eventually transferred to a remote computing device, where it is stored in memory. Suitable encryption algorithms known in the art or other suitable methods of encryption also can be used to protect the confidentiality of personally-identifiable information, such as first name, last name, and address, or other sensitive information.

Data may be stored in one or more databases or data warehouses in memory. The data storage component may reside local to and/or remote from the mobile computing device and may be configured to store one or more pieces of data for access by the mobile computing device and/or other components. Generally, a database is an organized body of information, such as a collection of data arranged for search and retrieval, and may include structured tables or records, multiple computer files, or a single file. In the illustrated example, a relational database product such as SQLServer, Oracle, or Access can be used, although any other suitable means for storing and retrieving data also may be used.

In a preferred embodiment of the invention, the computer program stored on the mobile device 1 is realized in form of an App or Application. An App, sometimes referred to as a mobile app or a mobile application, is a software application specifically designed to run on mobile devices such as smartphones and tablet computers. The App is downloadable into the storage medium 4 from a download server to which the mobile device 1 is connectable. The App may be adapted to a particular mobile operating system, such as Apple iOS, Google Android or Blackberry OS and distributed through known application distribution platforms.

It should thus be appreciated that “the App” hereinafter refers to the computer program stored on the storage medium 4 of the mobile device 1. The App can be executed by means of touching a particular icon displayed on the display 5 of the mobile device 1.

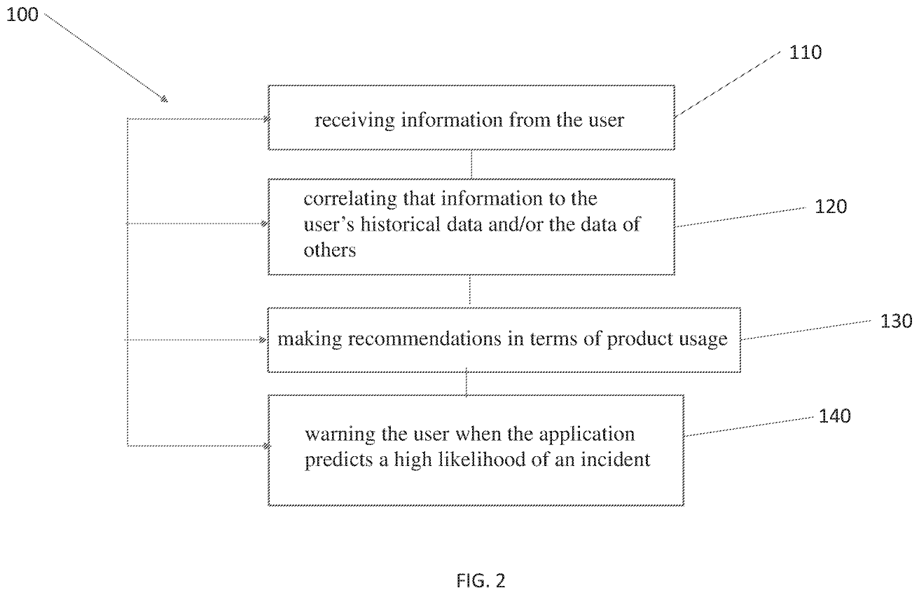

FIG. 2 represents the basic principles of the method 100. The method may include the steps of: receiving information from the user 110, correlating that information to the user's historical data and/or the data of others 120, making recommendations in terms of product usage 130, warning the user when the application predicts a high likelihood of an incident 140. The method may skip any one of these steps on any given day. For example, the method may not receive information from the user on a given day but may still make a recommendation or warn the user when it predicts a high likelihood of an incident.

The mobile computing device may prompt the individual to submit information. Input information collected from the consumer may be in response to questions in the form of a survey. The consumer's response to these questions may then be used to determine areas of improvement in the placement of the absorbent articles and correlations between absorbent article characteristics and the placement accounting for styles of undergarments. Consumers may be asked to submit surveys both prior and after using the product. Similarly, a survey request may occur at certain times while using the product. For example, a survey request may require submission of surveys every 3 hours for up to 12 hours. Alternatively, a survey request may request submission within a fixed time after the consumer considers the absorbent article stained. A survey request may also occur in response to the submission of video or image data by the consumer. Questions asked may be focused on, for example, length of time absorbent article was used, comfort and fit of absorbent article, perceived leakage, unintentional urine loss, location of fluid on the absorbent article or undergarment, depth of fluid on the absorbent article, stain size, or any other suitable questions. Furthermore, these questions may be asked of the consumer in any suitable fashion, for example, via the mobile computing device or the World Wide Web. Questions may also request the consumer's age, consumer's absorbent article preference, consumer's prior absorbent article use, what other feminine hygiene products the consumer has used or is currently using, consumer's description of her flow on her heaviest day of her period, how many absorbent articles in total does the consumer use in her period, time between changing absorbent articles on the consumer's heaviest day, any leaks while using an absorbent article on the consumer's heaviest day, what is the consumer's confidence that when the absorbent article is changed during the heaviest time that the absorbent article has been fully used, what is the level of confidence the consumer has regarding the absorbent article and her interaction with the absorbent article, and the consumer's impression of future concepts and ideas. Questions may also request information related to what types of undergarments the consumer uses, how often the consumer uses a particular type of undergarments, or how a consumer places the absorbent article in the undergarment.

The questions may be used to determine categories that may be identified as profile, segment, and/or classification. This separation into profile, segment, and/or classification may be produced in any suitable fashion, including but not limited to, questions asked of consumers, such as questions about habits, traits, personality, and/or responses to visual situations and/or images of the consumers. The profile, segment, and/or classification into which a particular consumer belongs to may be similarly performed. These questions, for example, may be based on habits, traits, personality, and/or responses to visual situations and/or images of the consumers. One system of question asking to classify consumers into profile, segment, and/or classification is psychographic segmentation.

Any suitable combination of either individual questions or groups of questions may be asked in order to collect sufficient information to provide the basis for developing correlations used to improve absorbent articles. The precise manner and wording chosen to collect this information from the consumer may vary depending on local custom, the comfort level consumers in a particular area may have in describing their flow characteristics, and the shades of meaning associated with terms which may be used in different parts of the world to collect the information desired. Questions may also include questions about pessary device use and pessary device types.

The questions may allow the application to create a user profile and understand the historical data of the user. Additionally, the application may receive input or information on a daily basis, every other day, or on a weekly basis.

The input or information may be used to predict future incidents or voiding. The input or information may include intake information on which the prediction of future voiding is based may comprise information relating to at least one and preferably a plurality of intakes of fluid and/or solid edible material by the product-wearer.

For each intake, the intake information may comprise a time indication indicating when the intake was made. The time indication may be an indication of the actual time of the intake or an indication of whether the intake was made during breakfast, lunch, dinner, etc. Furthermore, for each intake, the intake information may comprise a type indication indicating the type of the intake. The intake may be any type of intake, including but not limited to intake of liquid, (solid) food, or liquid or solid medicaments. Yet further, for each intake, the intake information may comprise an amount indication indicating the amount or size of the intake. The amount indication may be an indication of the actual volume or weight of the intake or it may be an indication of whether the intake was small, average or big in size.

The information on which the prediction of future voiding is based may also comprise data regarding the level of activity of the user, type of activity, the environment of the user such as, for example, environmental temperature, humidity levels, environmental altitude.

The information on which the prediction of future voiding is based may also comprise biometric data gathered by one or more devices. Devices include and are not limited to mobile phones, wearable heart rate monitors, smart watches with the capability of monitoring heart rate, sensors able to blood pressure, sensors able to measure strain of the skin surface, sensors able to measure peripheral capillary oxygen saturation, sensors able to measure bioimpedance, sensor able to measure odor through a VOC sensor, ultrasound sensors, acoustic sensors, myoelectric sensors, optical sensors, fluorescence, and/or moisture level on the skin. One or more sensors may be combined to give a set point of information. For example, bioimpedance and one of optical, fluorescence, and acoustic may be combined to approximate such measurements as, for example, bladder fullness. Additional sensors may include flow rate sensors that are placed at or near the urethra.

The information on which the prediction of future voiding is based may also comprise prior experience data correlations such as, for example, prior times of voiding including bathroom visits, the effect of a weekly activity on voiding versus other days, and/or correlation of voiding to distance from home. The use of global positioning services may be used to determine the user's life space or how far they venture from their home, their daily activity, and any changes in activity or life space. Additionally, the information on which predictions of future voiding is based on may also comprise prior calendar data of the user.

Global positioning services may further be used to make the user aware of their proximity to available products. For example, the application may inform the user that they are located near a store that carries a particular product. Additionally, the application may inform the user of the purchase price of the product at the nearby location and upload any relevant coupons for the recommended product.

For each insult, the voiding information may comprise at least a time indication indicating when the insult occurred. The time indication is preferably a parameter indicating the actual time of the insult. Furthermore, for each insult, the voiding information preferably comprises a type indication indicating the type of the insult, e.g. whether the insult was a urinary or fecal insult. Yet further, for each insult, the voiding information may comprise an amount indication indicating the amount urine or feces voided. The amount indication may be a parameter indicating an estimated or measured volume and/or weight of urine or feces voided, or a parameter indicating whether the insult was small, average or big in size.

For each insult, the voiding information may comprise additional data such as, for example, environmental data such as temperature and humidity levels, location data via global positioning services or other forms of identifying a location, biometric data such as heart rate.

The intake and voiding information may be obtained by the mobile device by having the product-wearer or his caregiver enter the information manually on the mobile device. Information may also be obtained passively by a device that is in communication with the application, such as, for example a Bluetooth enabled smart watch.

For example, once the application is opened, additional information may be obtained via communication with a separate Bluetooth device that is in communication with the mobile device. Such additional information, as previously disclosed above, may include biometric information such as heart rate, environmental conditions such as temperature, and physical location through the use of global positioning services.

The method may further comprise the use of a sensor in or on the absorbent article that provides information to the mobile application. The sensor may be in a durable panty form with built in absorbency. The intake and voiding information may be obtained from manual input of information on the mobile device relating to actual intakes and insults by the product-wearer. The information may be obtained by letting the product-wearer keep a daily digital log on the mobile device, wherein daily intakes of fluid and/or solid edible material as well as daily insults of urine and/or feces are registered by the product-wearer and used by the mobile phone to predict future voiding.

The method may further comprise a step of obtaining capacity information related to the capacity of the absorbent product. The product-related information may then be provided based on both the prediction of future voiding by the wearer and said capacity information. By providing the product-related information based on both the prediction and the capacity information, the product related information can be based on a relation between future urinary and/or fecal voiding of the product-wearer and the capacity of the product, which allows the product-related information to comprise well-founded recommendations as to the need of changing the product.

Preferably, the intake and voiding information is obtained by the mobile device 1 by letting the product-wearer keep a daily digital log on the mobile device, wherein daily intakes of fluid and/or solid edible material as well as daily insults of urine and/or feces are registered by the product-wearer. To this end, the App preferably comprises a user interface comprising information-collection means to which the intake and voiding information can be entered by the product-wearer. The information-collection means may comprise text fields, drop-down menus, option buttons etc. Preferably, the information-collection means are configured to allow the product-wearer to input intake information comprising information related to the time of the intake, the size of the intake (e.g. a volume or weight of the) and the type of the intake (e.g. fluid or solid). Similarly, the information-collection means are preferably configured to allow the product-wearer to input voiding information comprising information related to the time of urinary and/or fecal insults, the size of the insult (e.g. a volume or weight of urine and/or feces voided) and the type of the insult (e.g. urinary or fecal insult). The App is preferably configured to store the intake and voiding information in a database of the mobile device.

In other embodiments, the intake and/or the voiding information may, at least to some extent, be automatically obtained by the mobile device 1 based on information about the product-wearer. In this case the method may comprise the step of presenting, on the mobile, device, one or more questions about to the product-wearer, and to derive the intake and/or voiding information based on the answers to said questions. In addition to the information about the product-wearer, known statistical data on intake and/or voiding behavior may be used to derive the intake and/or voiding information. The questions may relate to the product-wearer's normal intake routines and/or the product-wearer's normal voiding behavior. They may also relate to the product-wearer himself and/or the health of the product-wearer. For example, the questions may include questions about when the product-wearer normally eats, drinks, urinates and defecates, and/or questions about the product wearer's age, gender, health, use of medicaments, etc. Information relating to the product-wearer's potential use of drugs for treatment of incontinence (e.g. the type and/or dosage of the drug) is of particular interest and may be used by the App to obtain relevant voiding information. The obtained information about the product-wearer can then be used to estimate the intake and/or the voiding information.

In other embodiments, the intake and/or the voiding information may be obtained automatically by the mobile device 1. For example, the mobile device may be configured to identify a food or liquid product automatically by means of a camera and suitable image recognition software, an RFID reader or a barcode scanner of the mobile device, and to obtain at least some of the intake information automatically from a product database stored on the mobile device 1 or an application server with which the mobile device can communicate. To this end, the absorbent product may be provided with means for facilitating automatic identification thereof, such as a barcode (e.g. a QR code) or an RFID tag. The voiding information may also be obtained automatically by the mobile device 1 through communication with an external insult monitoring device. An example of such an insult monitoring device is a wetness sensor arranged in the absorbent product and configured to communicate detection of urinary and/or fecal insults to the mobile device. Another example is an odour sensor configured to detect urinary and/or fecal insults through gas composition analysis and to communicate the detection of an insult to the mobile device. Alternatively, the method may be used with absorbent articles that are free of sensors.

The method may further include correlating that information to the user's historical data and/or the data of others. The method above may utilize the information gathered to personalize the usage experience for an individual or for a group or cohort. The method may, via the transformation of data, make recommendations to the user that are personally tailored to their needs and personal calendar of activities.

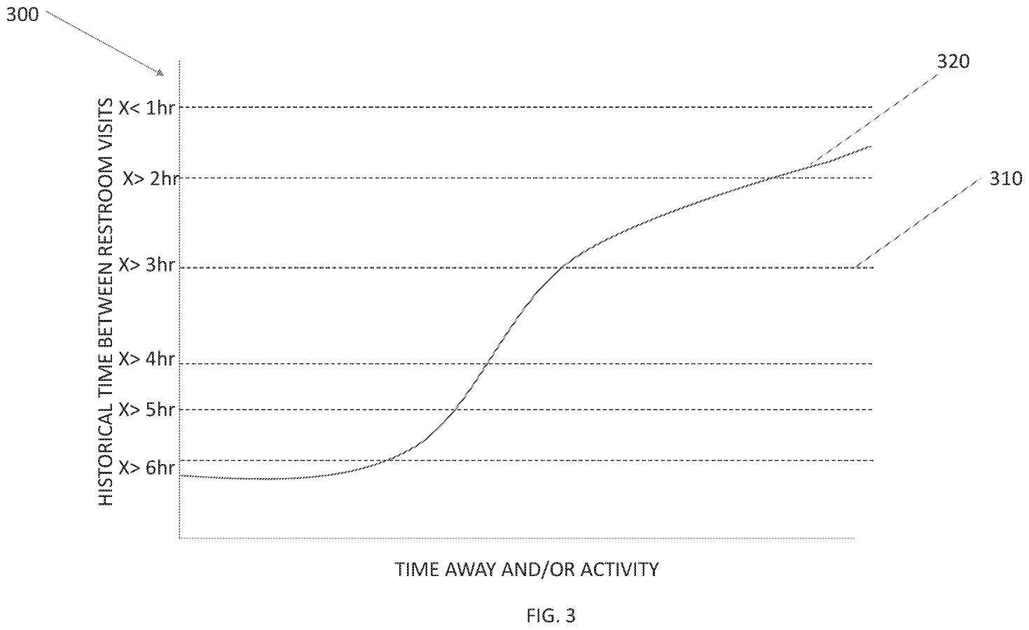

The application may utilize the inputs to create a personalized curve that can make product recommendations dependent upon the chosen activity or time away from home. The application may give product recommendations for a future activity or a given activity input by the user. As shown in FIG. 3, the application may create an individual curve for a user that is capable of recommending different product types and/or different levels of absorbency based upon the user's input including and not limited to using an absorbent disposable product, using an undergarment that contains an absorbent, or using an undergarment alone without an absorbent product; the activity located in the user's calendar; and/or the prior historical data of the user. The curve 320 of FIG. 3 is shown as a graph 300 having Historical time between restroom visits on the Y axis and Time away and/or Activity on the X axis. The dashed lines 310 break the curve into product segments. Each product segment may contain one or more products that may be recommended by the App. For example, if the user is to be gone for a period of time that places them in the steep part of the curve, they choose to use one or more products that fall within that product segment. The product segments may increase in absorbency as one moves up in the table along the Y axis. The individual curve may or may not show bands for each product to illustrate what products would be best for certain conditions. Product recommendations may include one of or a combination of pessary devices, liners, feminine hygiene pads, incontinence pads, incontinence pants, and durable absorbent products.

The method may transform inputs into insights and foresights and personalized behavioral interventions to manage and prevent incontinence. The transformation may be done through the use of an algorithm.

The algorithm may give an amount of weight to each of: sensor data, contextual information data, data from the database or historical data, daily communications added by the user, fluid balance based on inputs and outputs. Utilizing the information, the algorithm may give a recommendation regarding life space and product usage. The recommendation may include which products to use on a given day, how many products to take with them for the day, estimate timings of future bathroom usage, and/or estimate amount of time between future bathroom usage.

For example, the algorithm may account for the following:

[sensor]+[contextual information]+[algorithm/database]+[communication]+[user interface]+[fluid management]=Life space and confidence expansion

The method may transform inputs into insights and foresights and personalized behavioral interventions to manage and prevent incontinence. The transformation may be done through the use of artificial intelligence. Artificial intelligence may utilize the data and math (algorithm(s)) to uncover new information without human intervention. The algorithms are enabled to learn from the data provided, gather insights, and make prediction on previously unanalyzed data using information gathered.

The method may make recommendations in terms of product usage dependent upon the user's historical data and/or the inputs received for a given day or set of conditions. The product-related. information is preferably provided to the product-wearer through visual, audible and/or vibratory signaling on the mobile device 1. Instead, or in addition, it may be provided on a communication device, such as another mobile device or a computer, with which the mobile device can communicate, for example a communication device of a caregiver of the product-wearer. In the latter scenario, the product-related information may be provided to the communication device e.g. in form of a text message (sms) or an e-mail.

The method may predict a future voiding or incident based on intake and voiding information, historical data, and current environmental and user conditions, and providing recommendations as to the use of the absorbent product based on said prediction. The method may make use of the findings that daily intake of fluid and solid edible material and urinary and fecal behavior of the user can be used to predict future voiding behavior.

The method may further make use of the impact of different activities and or biometric data to predict future voiding behavior. The method further makes use of data gathered for a given cohort to make recommendations to a user when prompted. By registering or by other means obtaining information relating to the daily intake and voiding behavior of the wearer under average daily conditions and under other conditions, product-related information that facilitates the use of the absorbent product can be derived and provided to the wearer or a caregiver of the wearer. For example, the product-related information may comprise a recommendation to change the absorbent product before a certain time, displayed on the mobile device, or another communication device with which the mobile device can communicate. Additionally, for example, the product related information may recommend, via a mobile device, computer, personal electronic assistant, or other form of communication, to take a number of products such as, for example, one, two, three, or four products with the user based on that day's given activities. The day's given activities may be based on a prior set of data, an input on the day of by the user, or by connecting to a calendar on the user's mobile device or computer.

The method may change product recommendations over time depending on the information received and/or the changing behavior of the user. For example, the method may recognize an increase or decrease in incidents or an expansion in lifespace and therefore make new product recommendations. The method may change recommendations based on the daily activity such that one product is recommended on a particular day versus another dependent on the activities for that day. Through the use of the application, the user may choose to, over time, expand their lifespace or the distance and time they are willing to venture from their home due to a reduced fear of incidents and better control over their personal incontinence and product usage.

The method may allow the user to have products sent directly to the user based on the recommendations of the method. The products sent to the user may change over time as the user expands their lifespace or as the needs of the user change. The application may determine the changing needs of the user based on the data provided. The application may determine the changing needs via the use of algorithms and artificial intelligence.

Additionally, the application may create a simulator based upon the historical data of the user. The simulation may be used to anticipate the user's absorbent article needs under different conditions and/or over various time periods. The simulation may allow the user to adjust variable such as, for example, the amount of time away from home, the future location and distance to public restrooms, the level of activity for the day, current and future weight, current health conditions such as, for example, if the user has a stomach ache, a virus, or any other ailment, the intake of diuretics such as coffee or alcohol, and/or the intake of fluids and solids.

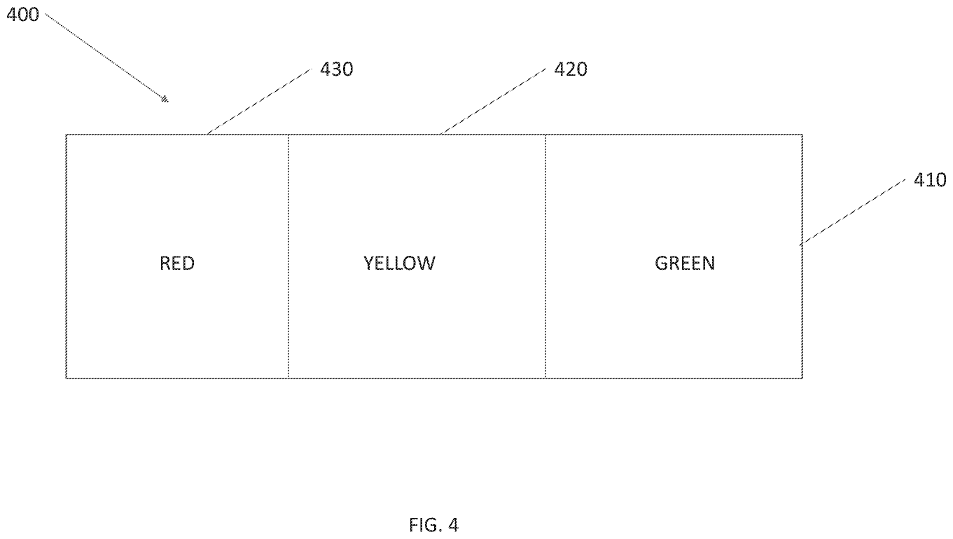

The method may warn the user when the application predicts a high likelihood of an incident. For example, as shown in FIG. 4, the historical data and the product recommendation may be used to create a “Confidence Indicator” 400. The Confidence Indicator allows the user to quickly determine their current level of risk regarding a possible incident. For example, if the indicator is green 410, it represents that, based on the user's individual curve and the given inputs for the day, the user is not at risk of an incident. If the indicator is yellow 420, it represents that the user may be at risk of an incident and that they should consider finding a bathroom facility in the near future. If the indicator is red 430, it means that the user should find a bathroom facility as soon as possible. The Confidence Indicator may work with the simulator to allow the user to determine a confidence level for different scenarios and with different products.

The mobile application may utilize changing colors to allow the user to know when a future incident may occur. For example, the application may include a signal that changes from green to yellow to red over time. If the user utilizes a bathroom and empties their bladder, the application may, via a sensor, recognize that the person has used a restroom and modify the current Confidence Interval. Additionally, the user may input the use of the bathroom, thereby allowing the Confidence Interval to modify.

The Application may warn the use via vibration, an email, or an alarm if the Confidence Interval is within a certain range or parameter. For example, the application may warn the user during the night if the application determines that the user may have an incident. The application may use the historical data and the confidence indicator level to determine when to contact the user via vibration, email, or an alarm. The user may choose to create a threshold level at which they want to be notified by the application. The user may choose, from the options provided, the means by which the application communicates once the threshold is reached.

The application may further use the historical data of the user and of the other members to allow any one user to know how their incontinence compares to others. The user may be allowed to compare based on gender, age, type of product worn. The user may create a reference point of their current standing in any one of the cohorts possible and see how they change over time. The application may give a user information about what products other people in their cohorts are using. The application may give a user information regarding what other users with a similar condition or in a similar cohort are using to manage their incontinence.

The application may use the historical data to offer a selection of products to a user. The selection may be individually tailored based on any of the historical inputs discussed above. The user may then choose to purchase the product selection or modify the product selection. Once purchased, the products may be sent to the user's home. The user may choose to subscribe to a group of products so that they arrive on a regular basis, such as, for example, weekly, biweekly, or monthly. The user may choose to subscribe to an auto selected group of product(s). The application may auto select the group of products based on the current historical data.

The mobile device utilizes a user interface in the form of a mobile application for user information input, and behavioral modification via notifications and coaching through the mobile application. The mobile application may create a feedback loop (computing/communication/interface) to minimize incontinence events, and ensure proper fluid management protection is in place for real time leakage risk predictions.

The mobile application or the method may utilize data and measure the response to those interventions (life space, amount and quantity of leaks) until a routine is established and leakage is minimized. Once fluid management is personalized optimized, the user may increase their life space increase along with their confidence.

The methods of the invention can be used with any suitable feminine hygiene article. Suitable absorbent articles include any type of structures, from a single absorbent layer to more complex multi layer structures. Certain absorbent articles typically include a fluid pervious topsheet, a backsheet, which may be fluid impervious and/or may be water vapour and/or gas pervious, and an absorbent element often called “core” comprised there between.

The topsheet may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be included of natural fibers (e.g., wood or cotton fibers), synthetic fibers (e.g., polymeric fibers such as polyester, polypropylene, or polyethylene fibers) or from a combination of natural and synthetic fibers. In one embodiment, the topsheet may be made of a hydrophobic material to isolate the wearer's skin from liquids which have passed through the topsheet. If the topsheet is made of a hydrophobic material, at least the upper surface of the topsheet is treated to be hydrophilic so that liquids will transfer through the topsheet more rapidly. This diminishes the likelihood that body exudates will flow off the topsheet rather than being drawn through the topsheet and being absorbed by the absorbent core. In one embodiment, the topsheet can be rendered hydrophilic by treating it with a surfactant. Suitable methods for treating the topsheet with a surfactant include spraying the topsheet material with the surfactant and immersing the material into the surfactant.

The topsheet can include an apertured formed film. Apertured formed films can be used for the topsheet because they are pervious to body exudates and yet non-absorbent and have a reduced tendency to allow liquids to pass back through and rewet the wearer's skin. Thus, the surface of the formed film which is in contact with the body remains dry, thereby reducing body soiling and creating a more comfortable feel for the wearer. Suitable formed films are described in U.S. Pat. No. 3,929,135, entitled “Absorptive Structures Having Tapered Capillaries”, issued to Thompson on Dec. 30, 1975; U.S. Pat. No. 4,324,246 entitled “Disposable Absorbent Article Having A Stain Resistant Topsheet”, issued to Mullane, et al. on Apr. 13, 1982; U.S. Pat. No. 4,342,314 entitled “Resilient Plastic Web Exhibiting Fiber-Like Properties”, issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, issued to Ahr, et al. on Jul. 31, 1984; and U.S. Pat. No. 5,006,394 “Multilayer Polymeric Film” issued to Baird on Apr. 9, 1991.

The absorbent core can be any absorbent member which is generally compressible, conformable, non-irritating to the wearer's skin, and capable of absorbing and retaining body fluids. The absorbent core may be manufactured in a wide variety of sizes and shapes (e.g., rectangular, hourglass, “T”-shaped, asymmetric, etc.) and from a wide variety of liquid-absorbent materials commonly used in disposable pull-on garments and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core may vary (e.g., the absorbent core may have varying caliper zones, a hydrophilic gradient, a superabsorbent gradient, or lower average density and lower average basis weight acquisition zones; or may include one or more layers or structures). Further, the size and absorbent capacity of the absorbent core may also be varied to accommodate wearers ranging from infants through adults. However, the total absorbent capacity of the absorbent core should be compatible with the design loading and the intended use of the absorbent article.

The absorbent core may include other optional components. One such optional component is the core wrap, i.e., a material, typically but not always a nonwoven material, which either partially or totally surrounds the core. Suitable core wrap materials include, but are not limited to, cellulose, hydrophilically modified nonwoven materials, perforated films and combinations thereof.

The backsheet can comprise a liquid impervious film. The backsheet can be impervious to liquids (e.g., body fluids) and can be typically manufactured from a thin plastic film. However, typically the backsheet can permit vapours to escape from the disposable article. In an embodiment, a microporous polyethylene film can be used for the backsheet. A suitable microporous polyethylene film is manufactured by Mitsui Toatsu Chemicals, Inc., Nagoya, Japan and marketed in the trade as PG-P.

One suitable material for the backsheet can be a liquid impervious thermoplastic film having a thickness of from about 0.012 mm (0.50 mil) to about 0.051 mm (2.0 mils), for example including polyethylene or polypropylene. Typically, the backsheet can have a basis weight of from about 5 g/m² to about 35 g/m². However, it should be noted that other flexible liquid impervious materials may be used as the backsheet. Herein, “flexible” refers to materials which are compliant and which will readily conform to the general shape and contours of the wearer's body.

The backsheet can be typically positioned adjacent the outer-facing surface of the absorbent core and can be joined thereto by any suitable attachment device known in the art. For example, the backsheet may be secured to the absorbent core by a uniform continuous layer of adhesive, a patterned layer of adhesive, or an array of separate lines, spirals, or spots of adhesive. Illustrative, but nonlimiting adhesives, include adhesives manufactured by H. B. Fuller Company of St. Paul, Minn., U.S.A., and marketed as HL-1358J. An example of a suitable attachment device including an open pattern network of filaments of adhesive is disclosed in U.S. Pat. No. 4,573,986 entitled “Disposable Waste-Containment Garment”, which issued to Minetola et al. on Mar. 4, 1986. Another suitable attachment device including several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Alternatively, the attachment device may include heat bonds, pressure bonds, ultrasonic bonds, dynamic mechanical bonds, or any other suitable attachment device or combinations of these attachment devices.

The backsheet may be additionally secured to the topsheet by any of the above-cited attachment devices.

The absorbent article may also include such other suitable features as are known in the art including, but not limited to, re-closable fastening system, lotion, acquisition layers, distribution layers, wetness indicators, sensors, elasticized waist bands and other similar additional elastic elements and the like, belts and the like, waist cap features, containment and aesthetic characteristics and combinations thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A method for monitoring incontinence and the use of an absorbent product worn by a wearer, the method comprising at least the steps of:
Obtaining information through the use of a mobile device,
predicting, by the mobile device, based on the information, a future voiding by the wearer,
providing, by the mobile device, product recommendations for a given activity.

2. The method of claim 1, wherein the absorbent article comprises a feminine hygiene pad, pantiliner, sanitary napkin, incontinence pad, incontinence pant, durable product with absorbent, or a pessary device.

3. The method of claim 1, wherein the intake information comprises information relating to one or more intakes of fluid and/or solid material by the wearer, and the voiding information comprises information relating to one or more urinary and/or fecal insults by the wearer.

4. The method of claim 1, wherein the method further comprises obtaining, by the mobile device, information about the wearer, wherein the information about the wearer comprises information selected from at least one of: normal intake routines of the wearer; normal voiding behavior of the wearer; age of the wearer; gender of the wearer; health of the wearer, and use of medicines by the wearer.

5. The method of claim 4, wherein the wherein the intake information comprises an indication of a point in time for one or more intakes, and the voiding information comprises an indication of a point in time for one or more insults of urine and/or feces.

6. The method of claim 5, wherein the method further comprises receiving information about a future activity by the user or passively by the user's calendar.

7. The method of claim 5, wherein the method further comprises giving input to the user suggesting a change in product, product usage, or product change frequency.

8. The method of claim 1, wherein information is received by the mobile device via Bluetooth from one or more sensors.

9. The method of claim 1, wherein the product-related information further comprises a recommendation to use a restroom facility prior to a set amount of time to reduce the potential for an incontinence incident.

10. The method of claim 1, wherein the method further comprises displaying a confidence indicator for a given product recommendation, wherein the confidence indicator changes over time to give.

11. A method for minimizing incidents of incontinence, the method comprising at least the steps of:
monitoring a user's bathroom habits and the use of an absorbent product,
obtaining, by means of a mobile device, intake information indicative of intake of fluid and/or solid edible material by the wearer,
obtaining, by means of one or more sensors, information regarding one or more of the user's environment, the user's daily activity, the user's calendar, and or the user's biometric data, and
providing product-recommendations for a future activity.

12. The method of claim 11, wherein the absorbent article comprises a feminine hygiene pad, pantiliner, sanitary napkin, incontinence pad, incontinence pant, durable product with absorbent, or a pessary device.

13. The method of claim 11, wherein the intake information comprises information relating to one or more intakes of fluid and/or solid material by the wearer, and the voiding information comprises information relating to one or more urinary and/or fecal insults by the wearer.

14. The method of claim 11, wherein the method further comprises obtaining, by the mobile device, information about the wearer, wherein the information about the wearer comprises information selected from at least one of: normal intake routines of the wearer; normal voiding behavior of the wearer; age of the wearer; gender of the wearer; health of the wearer, and use of medicines by the wearer.

15. The method of claim 14, wherein the wherein the intake information comprises an indication of a point in time for one or more intakes, and the voiding information comprises an indication of a point in time for one or more insults of urine and/or feces.

16. The method of claim 15, wherein the method further comprises giving input to the user suggesting a change in product, product usage, or product change frequency.

17. The method of claim 11, wherein the product recommendation is based on historical data of the user.

18. The method of claim 11, wherein information is received by the mobile device via Bluetooth from one or more sensors.

19. The method of claim 11, wherein the product-related information further comprises a recommendation to use a restroom facility prior to a set amount of time to reduce the potential for an incontinence incident.

20. The method of claim 11, wherein the method further comprises displaying a confidence indicator for a given product recommendation, wherein the confidence indicator changes over time to give.
